(19) United States
(12) Patent Application Publication
(10) Pub. No.: US 2022/0139265 A1
(43) Pub. Date: May 5, 2022

(54) SURGICAL SIMULATOR AND METHODS OF USE

(71) Applicant: The Board of Supervisors of Louisiana State University and Agricultural and Mechanical College, Baton Rouge, LA (US)

(72) Inventor: Michael E. Dunham, New Orleans, LA (US)

(21) Appl. No.: 17/621,106

(22) PCT Filed: Jun. 22, 2020
(86) PCT No.: PCT/US2020/038913

§ 371 Date: Dec. 20, 2021

Related U.S. Application Data
(60) Provisional application No. 62/864,987, filed on Jun. 21, 2019.

(51) Int. Cl. G09B 23/34; G09B 9/00

(52) U.S. Cl. CPC G09B 23/34; G09B 9/00

(57) ABSTRACT

Provided are bench model surgical simulation devices that incorporate capacitance sensing technology. In embodiments, the surgical simulation devices objectively evaluate operator proficiency and improve trainee performance with regard to an underlying surgical procedure. This disclosure further provides for systems and methods of surgical simulation and evaluation of operator proficiency.

This application claims priority from U.S. Provisional Application No. 62/864,987, filed on Jun. 21, 2019, the entire contents of which are incorporated herein by reference.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE DISCLOSURE

This disclosure relates to bench model surgical simulation devices and associated systems and methods of use.

### BACKGROUND

Tympanostomy tube insertion is the most commonly performed surgical procedure in children. Beyond basic soft tissue handling and suturing, it is one of the first surgical skills acquired by otolaryngology residents. Otologic surgery is highly specialized and technically challenging and trainees often struggle to gain proficiency working through the ear canal under a microscope. Inexperienced surgeons are more likely to encounter inaccurate tube placement, canal injury, troublesome bleeding and prolonged anesthesia.

### SUMMARY OF THE DISCLOSURE

Aspects of the invention are directed towards a surgical simulator comprising a surgical replica configured to approximate a surgical tissue or a surgical field. The surgical simulator can further include a capacitance sensor with at least one sensor surface. In embodiments, the surgical simulator includes a processing system communicatively linked to the capacitance sensor and configured to provide an operator proficiency score. The surgical simulator can be configured to simulate a surgical procedure and assess an operator's proficiency with the surgical procedure.

In embodiments, the sensor surface of the surgical simulator is configured to detect contact with an electrical conductor. In certain embodiments, the electrical conductor comprises a surgical instrument.

In embodiments, the surgical simulator can be configured to simulate surgeries in the field of general surgery, otolaryngology, neurosurgery, gastroenterology, urology, cardiovascular surgery, oral surgery, pediatric surgery, plastic surgery, orthopaedic surgery, or cardiothoracic surgery, dentistry, podiatry, or any a combination thereof.

In embodiments, the surgical procedure comprises myringotomy, tympanostomy tube insertion, endoscopic sinus surgery, skull base surgery, laryngeal surgery other types of ear surgery or a combination thereof.

In embodiments, the at least one sensor surface of the surgical simulator comprises a copper foil, a conductive cloth, conductive paint, or a combination thereof. The at least one sensor surface can be at least partially integrated within or coated upon the surgical replica.

In embodiments, the surgical simulator includes a display screen, wherein the display screen is configured to display the operator proficiency score.

In certain embodiments, the surgical tissue comprises a human ear. The surgical replica can comprise an artificial human ear. The artificial human ear can comprise a middle ear section and an outer ear section. Certain artificial human ear embodiments comprise an auricle, an external auditory canal, a tympanic cavity, a tympanic membrane, or a combination thereof. The external auditory canal further can further include a cartilaginous-type canal, a bony-type canal, or a combination thereof. In embodiments, the at least one sensor surface is integrated within the auricle, the external auditory canal, the tympanic cavity, the tympanic membrane, or a combination thereof. The tympanic membrane can be configured to be replaced between simulations. The tympanic membrane can comprise a thickness of about 0.5 mm to about 1.5 mm. The tympanic membrane can be a flexible film that further comprises wax and polyolefins.

The invention is further directed towards a method of simulating a surgical procedure. In embodiments, the method comprises simulating a surgical procedure using a surgical simulator as described in any one or more of the various exemplary embodiments disclosed herein. The method can include determining the total amount of time required to complete the surgical procedure. In embodiments, the method comprises determining the total amount of sensor contact time. By way of example, sensor contact time can be the amount of time that an electrical conductor was in contact with the sensor surface during the surgical procedure.

The method can further include providing an operator proficiency score. In embodiments, the proficiency score is inversely proportional to the total amount of time required to complete the surgical procedure, the total amount of sensor contact time, or a combination thereof.

In embodiments, the method can include the step of displaying the operator proficiency score on a display screen.

Aspects of the invention are further directed towards a system for simulating a surgical procedure. The system includes the surgical simulator as described in any one or more of the various exemplary embodiments disclosed herein.

The system can further include software configured to calculate the operator proficiency score. In embodiments, the system includes a display screen configured to display an operator proficiency score.

In embodiments, the system is configured to determine running time and sensor contact time.

In embodiments, the system further includes a microcontroller, wherein the microcontroller comprises a timer, an interface to the sensors, an output to the display, or a combination thereof. The microcontroller can be configured to detect the total time required to complete the surgical procedure, the number of contacts between an electrical conductor and the sensor surface, the total amount of time that the electrical conductor contacts the sensor surface, or a combination thereof. The microcontroller can be configured to control a procedure start time, to control a procedure stop time, reset the system, or a combination thereof.

In embodiments, the system is configured to track instrument placement accuracy.

### BRIEF DESCRIPTION OF THE DRAWINGS

The present disclosure is focused on bench model surgical simulation devices and associated systems and methods of use. For example, the unique surgical simulator and associated systems and methods were designed to realistically simulate myringotomy with tympanostomy tube insertion, as summarized in the following figures. It is to be understood that this disclosure is not limited to particular embodiments described, and as such may, of course, vary. Further aspects of the present disclosure will be readily appreciated upon review of the detailed description of its various embodiments, described below, when taken in conjunction with the accompanying drawings.

The patent or application file contains at least one drawing executed in color. Copies of this patent or patent application publication with color drawing(s) will be provided by the Office upon request and payment of necessary fee.

FIG. 1 is a photograph illustrating a top view of the assembled surgical simulator under one embodiment.

FIG. 2 is a photograph showing the conceptual design of the surgical simulator under another embodiment.

FIG. 3 illustrates CT scan images (top left, top right, and bottom left) and a computer reconstruction of the external ear (bottom right) and medial external auditory canal.

FIG. 4 is a table showing the external auditory and tympanic cavity dimensions under one embodiment.

FIG. 5 shows various 3D printed parts and tympanic membrane of the surgical simulator under one embodiment.

FIG. 6 provides a breadboard view of the sensor and scoring system in a prototypic embodiment.

FIG. 7 is a schematic representation of the sensor and scoring system under one exemplary embodiment.

FIG. 8 shows an exemplary application flow diagram.

FIG. 9 is a side perspective photographic view of the FIG. 1 embodiment in use by a trainee. The photograph shows a demonstration of the surgical simulator with a photomicrograph of the simulated myringotomy in the bottom right corner.

FIG. 10 provides a schematic representation of the capacitive touch sensing employed in various exemplary embodiments.

FIG. 11 provides a top photographic view of a functioning sensor and scoring system prototype under one embodiment.

FIG. 12 is an alternate circuit schematic under one embodiment.

FIG. 13 shows the program flow under one exemplary embodiment.

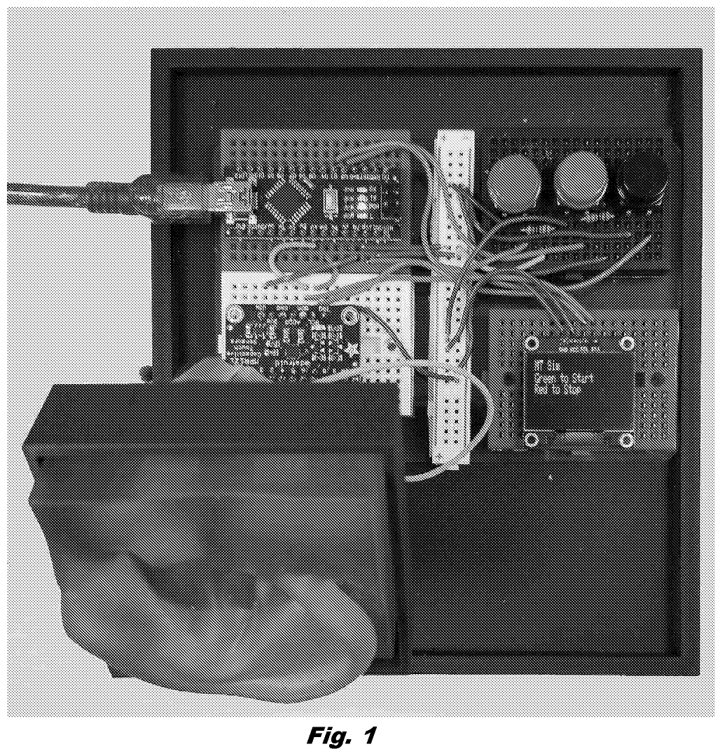

### DETAILED DESCRIPTION OF THE DISCLOSURE

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the advantageous methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, toxicology, engineering, mechanical engineering, electrical engineering, computer programming, computer engineering, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. patent law and can mean “includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein.

Prior to describing the various embodiments, the following definitions are provided and should be used unless otherwise indicated.

### Definitions

As used interchangeably herein, “subject,” “individual,” or “patient,” can refer to a vertebrate, preferably a mammal, more preferably a human. In certain embodiments, “subject,” individual,” or “patient” refers to a reptile. Mammals include, but are not limited to, murines, simians, humans, farm animals, sport animals, and pets. The term “pet” includes a dog, cat, guinea pig, mouse, rat, rabbit, ferret, snake, turtle, lizard, bird, and the like. The term farm animal includes a horse, sheep, goat, chicken, pig, cow, donkey, llama, alpaca, turkey, and the like.

The word “user,” “trainee,” or “operator” as used interchangeably herein, can refer to any individual attempting to become familiar or more familiar with a surgical procedure. A user of the device can include an undergraduate student, a medical student, a medical assistant, a nursing assistant, a resident, a physician's assistant, a nurse, a dentist, an orthodontist, an emergency medical technician, a veterinarian, a veterinary student, a surgeon, an optometrist, an obstetrician, or any other individual using the device or practicing the systems and methods disclosed herein.

### Surgical Simulation Devices and Methods of Use

The present disclosure is focused on bench model surgical simulation devices and associated systems and methods of use. For example, the unique surgical simulator and associated systems and methods were designed to realistically simulate surgical procedures as described herein. It is to be understood that this disclosure is not limited to particular embodiments described, and as such may, of course, vary.

The current method used to train medical professionals how to perform many surgical procedures is for the trainee to first watch an experienced professional perform a surgical procedure, and then have the trainee attempt the surgical procedure. For certain procedures, significant amount of practice is required in order to become proficient at safely and efficiently performing the procedure. Current simulators may be used for this practice, but such simulators are considered virtual simulators and do not utilize real surgical equipment or realistically mimic the subject's tissue.

Bench models can be physical replicas of the surgical field that are intended to simulate the tissue interactions with the instruments used for the corresponding in-vivo procedure. Advances in affordable 3D printing have recently facilitated the development of these types of models. However, current benchtop models have drawbacks as well. For example, they don't allow the user to detect misplaced implants and/or surgical mistakes. Thus, current benchtop models do not accurately reflect the proficiency of the user.

In various exemplary embodiments, the present disclosure provided bench model devices for simulation of a surgical procedure. In some embodiments, the device is engineered for realistic simulation of a surgical procedure.

The surgical simulator can comprise a sensing system designed to track instrument placement accuracy. The sensing system includes at least one sensor. In embodiments, the sensing system comprises more than one sensor. The sensing system can comprise up to 100 sensors. In embodiments, the sensing system comprises between 1 and 100 sensors, inclusive. The number of sensors in the sending system can range from 1 and 50 sensors. In certain embodiments, the system comprises between 1 and 25 sensors. In certain embodiments, the system comprises between about 1 and 10 sensors. In embodiments, the sensing system comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 sensing surfaces. The sensing system can comprise over 100 sensors. In embodiments, the sensing system comprises up to about 25 sensors, about 50 sensors, about 75 sensors, about 100 sensors, about 125 sensors, about 150 sensors, about 175 sensors, about 200 sensors, about 225 sensors about 250 sensors, about 275 sensors, about 300 sensors, about 325 sensors, about 350 sensors, about 375 sensors, about 400 sensors, about 425 sensors, about 450 sensors, about 475 sensors, or about 500 sensors, In embodiments, the sensing system can comprise up to 1000 sensors. Each sensor can comprise a sensor surface. In embodiments, the sensor surface comprises a conductive material that is incorporated within the sensing system. The sensor surface can be configured to detect errant contact by surgical instruments made during use of the surgical simulator. The sensing system can employ any of various conductive materials to detect instrument contact. Conductive materials can include any material that permits the flow of an electrical current. Exemplary conductive materials include metals, electrolytes, superconductors, semiconductors, plasmas, graphite, conductive polymers, or any other conductive material known in the art. In embodiments, the conductive material comprises copper foil, conductive cloth, conductive paint, or a combination thereof.

In embodiments, the conductive material can be placed into the simulated surgical tissue.

The surgical simulator can be provided with embedded software that is configured to grade the user or trainee. The systems and methods described here in can quantitatively measure the user's skill.

In embodiments, the surgical simulator compromises capacitance sensing technology that can measure instrument accuracy. In various embodiments, the surgical simulator is designed for use with actual surgical instruments

The surgical simulator can include software designed to objectively evaluate operator proficiency. The surgical simulator can be configured to improve trainee performance on a particular surgical procedure.

The surgical simulator can comprise a scoring system, such as a scoring system in communication with a sensor. For example, the scoring system can track parameters such as duration of surgery, accuracy of implant placement, or errant contact by surgical instruments to determine whether a user is or is not proficient in a certain surgical procedure.

In embodiments, the surgical simulator includes a surgical replica, which provides an anatomically accurate representation of surgical tissue or a surgical field. The surgical replica can be designed to approximate a variety of surgical tissues. The surgical replica can provide a three-dimensional model of surgical tissue. The surgical tissue can comprise the human body or any discrete portions thereof. Exemplary surgical tissues include, but are not limited to bone/joint, breast, lymphatic, cardiovascular, vascular, renal, genital, skin, urogenital, endocrine, respiratory, gastrointestinal, nervous system, or ear, nose, throat, or musculoskeletal. In embodiments, simulated surgical tissue comprises a nasal cavity, paranasal sinuses, a pharynx, a larynx, a central nervous system, an eye, a respiratory tract, a chest, a heart, a spine, extremities, a genitourinary tract, or a combination thereof. The surgical simulator mimic surgical tissue of a subject of any age. The subject can be an infant, a child, an adolescent, an adult, an elderly adult, or any combination thereof. In embodiments, the surgical simulator is custom designed according the surgical tissue of a specific subject.

The surgical replica can be designed to mimic the corresponding surgical tissue as closely as possible. This includes parameters that influence the look, feel, or texture of the simulated surgical tissue. These parameters include, but are not limited to, the thickness, hardness, elasticity, density, shape, size, or any other parameter or combination of parameters that contributes to the look and feel of the surgical tissue. In embodiments, the physical properties of the material used to mimic the parameters of the surgical tissue used can be determined and vary on a tissue by tissue basis.

The surgical simulation device can be configured to simulate any surgical procedure that can be performed on a subject. The simulator can be configured to simulate veterinary as well as human surgical procedures. The surgical procedures can include ectomies, ostomies, otomies, or a combination thereof. In embodiments, the surgical procedure comprises surgery in any one or more of the following fields: general surgery, dermatology, otolaryngology, neurosurgery, gastroenterology, urology, cardiovascular surgery, oral surgery, pediatric surgery, plastic surgery, orthopaedic surgery, and cardiothoracic surgery. Specific examples of surgical procedures include, but are not limited to, bursectomy, amputation, hemicorporectomy, hemipelvectomy, decompressive craniectomy, hemispherectomy, anterior temporal lobectomy, hypophysectomy, amygdalohippocampectomy, laminectomy, corpectomy, facetectomy, ganglionectomy, sympathectomy/endoscopic thoracic sympathectomy, neurectomy, nerve transfer, stapedectomy, mastoidectomy, photorefractive keratectomy, trabeculectomy, iridectomy, vitrectomy, glossectomy, esophagectomy, gastrectomy, appendectomy, proctocolectomy, colectomy, hepatectomy, cholecystectomy, pancreatectomy/pancreaticoduodenectomy, rhinectomy, laryngectomy, pneumonectomy, hypophysectomy, thyroidectomy, parathyroidectomy, adrenalectomy, pinealectomy, nephrectomy, cystectomy, tonsillectomy, adenoidectomy, thymectomy, splenectomy, lymphadenectomy, adenectomy, cervicectomy, clitoridectomy, hysterectomy, myomectomy, oophorectomy, salpingectomy, salpingoophorectomy, vaginectomy, vulvectomy, gonadectomy, orchiectomy, penectomy, posthectomy, prostatectomy, varicocelectomy, vasectomy, lumpectomy, mastectomy, coccygectomy, ostectomy, femoral head ostectomy, astragalectomy, discectomy, synovectomy, embolectomy, endarterectomy, frenectomy, ganglionectomy, gingivectomy, lobectomy, myomectomy, panniculectomy, pericardiectomy, gastrostomy, percutaneous endoscopic gastrostomy, gastroduodenostomy, gastroenterostomy, ileostomy, jejunostomy, colostomy, cholecystostomy, hepatoportoenterostomy, nephrostomy, ureterostomy, cystostomy, suprapubic cystostomy, urostomy, ventriculostomy, acryocystorhinostomy, amniotomy, clitoridotomy, hysterotomy, hymenotomy, episiotomy, meatotomy, nephrotomy, craniotomy, pallidotomy, thalamotomy, lobotomy, bilateral cingulotomy, cordotomy, rhizotomy, laminotomy, foraminotomy, axotomy, vagotomy, myringotomy, radial keratotomy, myotomy, tenotomy, fasciotomy, escharotomy, arthrotomy, tendon transfer, myotomy, heller myotomy, pyloromyotomy, anal sphincterotomy, lateral internal sphincterotomy, sinus surgery, sinusotomy, laryngoscopy, hysterectomy, cricothyrotomy, bronchotomy, thoracotomy, thyrotomy, tracheotomy, cardiotomy, phlebotomy, arteriotomy, and venotomy. In embodiments, the surgical procedure comprises laparotomy myringotomy and tympanostomy tube insertion.

The surgical stimulation device allows the user to use real surgical instruments (rather than, for example, joysticks or handheld wireless devices) while simulating any surgical procedure that can be performed on a subject.

The surgical stimulation device can measure time or duration of procedure, errant contact by surgical instrument, and instrument placement accuracy.

In one embodiment, the surgical simulation device comprises a bench model engineered for realistic simulation of myringotomy with tympanostomy tube insertion performed using an operating microscope. In embodiments, the sensing system tracks instrument placement accuracy and allows embedded software to grade the user and validate the system.

In embodiments, the surgical simulation device comprises a capacitance sensor, a microcontroller, and a surgical replica. The surgical simulation device can comprise a plurality of capacitance sensors or a capacitance sensing system. In certain embodiments, the capacitance sensor or capacitance sensing system is wired to or disposed upon relevant sites on the surgical replica, and the micro-controller registers and tracks instrument contact with the sensor or sensing system. The surgical simulation device and system can be configured to track instrument contact with specific sites on the surgical replica.

In embodiments, the surgical simulator and the systems and methods disclosed herein can be communicatively coupled with computer networks, computing devices, mobile devices, or combinations thereof. Under certain embodiments, the systems and methods disclosed herein may utilize the communicative coupling to relay data collected from the sensors. Such data can include, for example, the operator proficiency, the total amount of time required to complete the surgical procedure, the total amount of sensor contact time, the total number of sensor contacts, the location of the each sensor contact, or a combination thereof.

The communicative coupling can be accomplished through one or more wireless communications protocols. The communicative coupling may comprise a wireless local area network (WLAN). A WLAN connection may implement WiFi™ communications protocols. Alternatively, the communicative coupling may comprises a wireless personal area network WPAN. A WPAN connection may implement Bluetooth™ communications protocols.

Embodiments can comprise a data port for relaying data to the mobile device or other computing device. The data port may be a USB connection or any other type of data port. The data port allows for a wired communication between the surgical simulation device and separate computing devices. The data port may be used alone or in combination with the wireless communications protocols of the surgical simulation device described above.

Computer networks suitable for use with the embodiments described herein include local area networks (LAN), wide area networks (WAN), Internet, or other connection services and network variations such as the world wide web, the public internet, a private internet, a private computer network, a public network, a mobile network, a cellular network, a value-added network, and the like. Computing devices coupled or connected to the network may be any microprocessor controlled device that permits access to the network, including terminal devices, such as personal computers, workstations, servers, mini computers, main-frame computers, laptop computers, mobile computers, palm top computers, hand held computers, mobile phones, TV set-top boxes, or combinations thereof. The computer network may include one of more LANs, WANs, Internets, and computers. The computers may serve as servers, clients, or a combination thereof.

One or more components of the systems and methods described herein and/or a corresponding interface, system or application to which the systems and methods described herein are coupled or connected includes and/or runs under and/or in association with a processing system. The processing system includes any collection of processor-based devices or computing devices operating together, or components of processing systems or devices, as is known in the art. For example, the processing system can include one or more of a portable computer(s), portable communication device operating in a communication network, a network server, or a combination thereof. The portable computer can be any of a number and/or combination of devices selected from among personal computers, personal digital assistants, portable computing devices, and portable communication devices, but is not so limited. The processing system can include components within a larger computer system.

The processing system of an embodiment includes at least one processor. The term “processor” as generally used herein refers to any logic processing unit, such as one or more central processing units (CPUs), digital signal processors (DSPs), application-specific integrated circuits (ASIC), etc. The processor can be disposed within or upon a single chip. The processing system can further include at least one memory device or subsystem. The processing system can also include or be coupled to at least one database. The processor and memory can be monolithically integrated onto a single chip, distributed among a number of chips or components, and/or provided by some combination of algorithms. The systems and methods described herein can be implemented in one or more of software algorithm(s), programs, firmware, hardware, components, circuitry, in any combination.

The components of any system that include the systems and methods described herein can be located together or in separate locations. Communication paths couple the components and include any medium for communicating or transferring files among the components. The communication paths include wireless connections, wired connections, and hybrid wireless/wired connections. The communication paths also include couplings or connections to networks including local area networks (LANs), metropolitan area networks (MANs), wide area networks (WANs), wireless personal area networks (WPANs), proprietary networks, interoffice or backend networks, and the Internet. Furthermore, the communication paths include removable fixed mediums like floppy disks, hard disk drives, and CD-ROM disks, as well as flash RAM, Universal Serial Bus (USB) connections, RS-232 connections, telephone lines, buses, and electronic mail messages.

Aspects of the systems and methods or surgical simulation described herein may be implemented as functionality programmed into any of a variety of circuitry, including programmable logic devices (PLDs), such as field programmable gate arrays (FPGAs), programmable array logic (PAL) devices, electrically programmable logic and memory devices and standard cell-based devices, as well as application specific integrated circuits (ASICs). Some other possibilities for implementing aspects of the systems and methods pf surgical simulation described herein include: microcontrollers with memory (such as electronically erasable programmable read only memory (EEPROM)) or without memory, embedded microprocessors, firmware, software, etc. Furthermore, aspects of the systems and methods of surgical simulation described herein may be embodied in microprocessors having software-based circuit emulation, discrete logic (sequential and combinatorial), custom devices, fuzzy (neural) logic, quantum devices, and hybrids of any of the above device types. Of course the underlying device technologies may be provided in a variety of component types, e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, etc.

It should be noted that any system, method, and/or other components disclosed herein may be described using computer aided design tools and expressed (or represented), as data and/or instructions embodied in various computer-readable media, in terms of their behavioral, register transfer, logic component, transistor, layout geometries, and/or other characteristics. Computer-readable media in which such formatted data and/or instructions may be embodied include, but are not limited to, non-volatile storage media in various forms (e.g., optical, magnetic or semiconductor storage media) and carrier waves that may be used to transfer such formatted data and/or instructions through wireless, optical, or wired signaling media or any combination thereof. Examples of transfers of such formatted data and/or instructions by carrier waves include, but are not limited to, transfers (uploads, downloads, e-mail, etc.) over the Internet and/or other computer networks via one or more data transfer protocols (e.g., HTTP, FTP, SMTP, etc.). When received within a computer system via one or more computer-readable media, such data and/or instruction-based expressions of the above described components may be processed by a processing entity (e.g., one or more processors) within the computer system in conjunction with execution of one or more other computer programs.

Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### EXAMPLES

### Example 1

### ABSTRACT

### Objectives

Create a bench model device engineered for realistic simulation of myringotomy and tympanostomy tube insertion.

The system can include the following components:

1. 3D printed auricle, external auditory canal and tympanic cavity.

2. A capacitive sensor system integrated with the bony external canal and tympanic cavity that detects instrument contact.

3. A programmable microcontroller and supportive electronics with a timer and sensor interface.

4. Software to monitor the operating time and detect sensor interactions

### Results

Students and residents can practice tympanostomy tube insertion on a realistic simulator with quantitative measures of operator skill. The integrated capacitive sensing system provides a sensitive measure of instrument placement accuracy.

### Conclusions

MTSim is the first surgical simulator to incorporate capacitance sensing technology to measure instrument accuracy and software to objectively evaluate operator proficiency. Initial system validation shows that the simulator correlates with user experience. MTSim can improve trainee performance for myringotomy and tympanostomy tube insertion.

### INTRODUCTION

Tympanostomy tube insertion is the most commonly performed surgical procedure in children.¹ Beyond basic soft tissue handling and suturing, it is one of the first surgical skills acquired by otolaryngology residents. Otologic surgery is highly specialized and technically challenging and trainees often struggle to gain proficiency working through the ear canal under a microscope. Severe complications due to technical error are rare. However, inexperienced surgeons are more likely to encounter inaccurate tube placement, canal injury, troublesome bleeding and prolonged anesthesia.² Efficient surgical tympanostomy tube placement requires practice.³

Advances in computer technology and the commercialization of 3D printing have enabled the development of simulation for many surgical procedures. Published reports of surgical simulation include applications in general surgery, otolaryngology, neurosurgery, gastroenterology, urology and cardiovascular surgery.^{4, 5, 6}

Surgical simulators can be classified into three categories—bench models, animal and human cadavers and virtual reality. Animals and human cadavers are useful for training courses and anatomical dissection, but they are difficult to acquire on a regular basis and are not reusable. Recently, most surgical simulation investigators have focused on virtual reality (VR) systems.^{7, 8, 9} The VR movement has been aided by advances in graphic processing unit (GPU) technology, computer graphics software and virtual reality hardware including 3D visualization and haptic feedback devices. While VR is promising for some procedures, resemblance to corresponding in-vivo procedures and validated effectiveness is lacking in many cases.¹⁰

Bench models are physical replicas of the surgical field that are intended to simulate the tissue interactions with the instruments used for the corresponding in-vivo procedure. Advances in affordable 3D printing have recently facilitated the development of these types of models.¹¹

The device described in this report is a bench model engineered for realistic simulation of myringotomy with tympanostomy tube insertion performed using an operating microscope. A particularly innovative feature is a sensing system that tracks instrument placement accuracy and allows embedded software to grade the user and validate the system.

Specific project objectives include:

5. Construct a human ear model including the middle and outer ears from materials that realistically simulate human tissue.

6. Incorporate a sensor system capable of detecting and measuring instrument contact with the medial external auditory canal and tympanic cavity.

7. Incorporate a programmable microcontroller to monitor procedure run time and sensor contact and calculate the operator's statistics.

8. Use a modular design with a disposable tympanic membrane to allow easy reassembly between simulations.

The system can comprise any one or more of the following components (or any combination thereof):

9. A 3D printed auricle and cartilaginous external canal.

10. A 3D printed bony external auditory canal that articulates with the cartilaginous canal laterally and the tympanic cavity medially.

11. A 3D printed tympanic cavity.

12. A simulated tympanic membrane that can be replaced between procedures.

13. A capacitive sensor system integrated with the bony external canal and tympanic cavity.

14. A microcontroller with a timer and an interface to the sensors and an output display.

15. Housing for the system.

16. Software to monitor the operating time, detect sensor interactions and compile the user statistics.

### Design of One Embodiment

One embodiment was completed using a polyurethane 3D print of an auricle with a wooden tubular facsimile of the external auditory canal. Clear adhesive tape was used to simulate the tympanic membrane. This was affixed to the external auditory canal by a second section of wooden tubing. We tested the embodiment using a smart phone for visualization and concluded that the design was feasible (FIG. 2).

### 3D Modeling

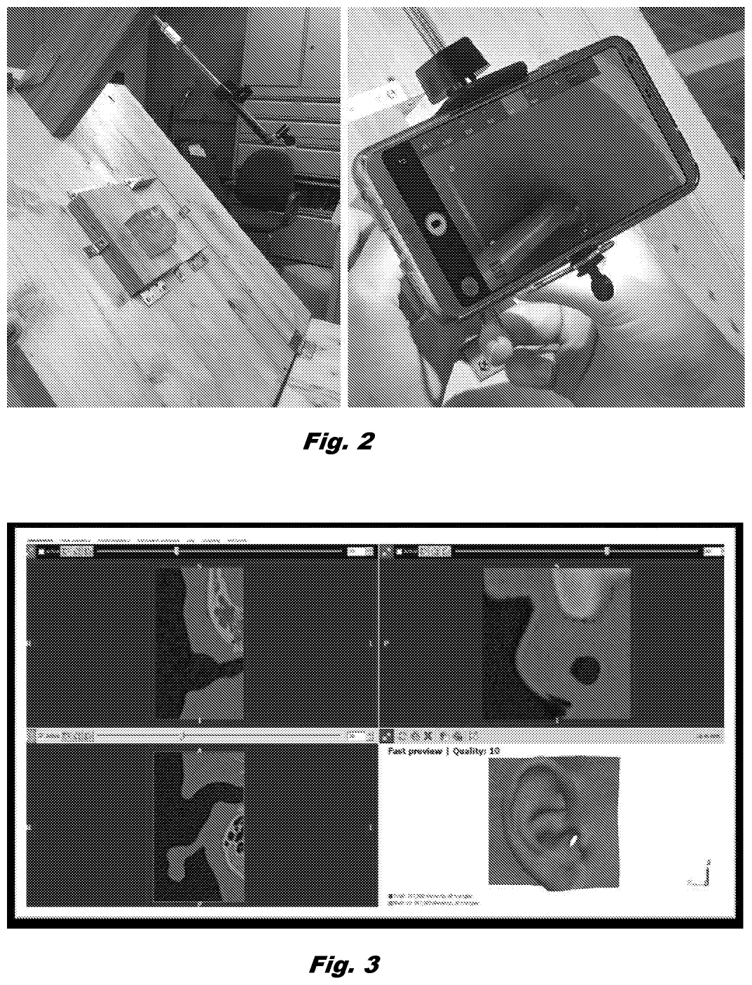

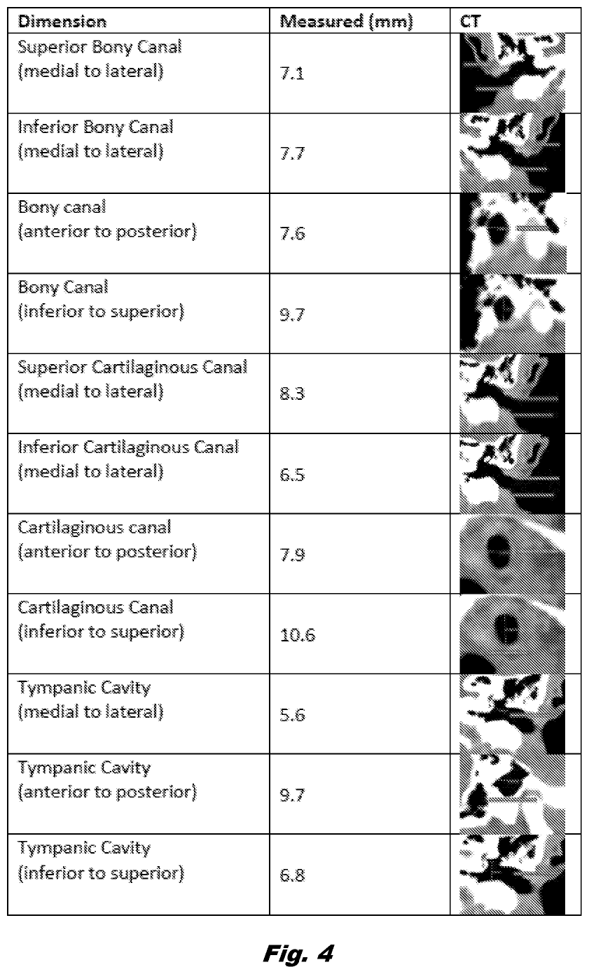

A right external ear, peri-auricular tissues and cartilaginous external auditory canal were reconstructed in software from the craniofacial CT scan of a 10-year-old female (FIG. 3). The scan was performed for indications other than ear disease. The Louisiana State University Health Sciences Center IRB granted an exemption for the use of the single, de-identified imaging study. The CT scan was imported into ScanIP image-processing and medical modeling software (Synopsys, Mountain View, Calif.) on a Dell Precision workstation. The modeling software created a 3-dimensional surface model that was saved in stereolithographic format. Relevant measurements of the external auditory canal and tympanic cavity acquired from the CT scan are shown in FIG. 4.

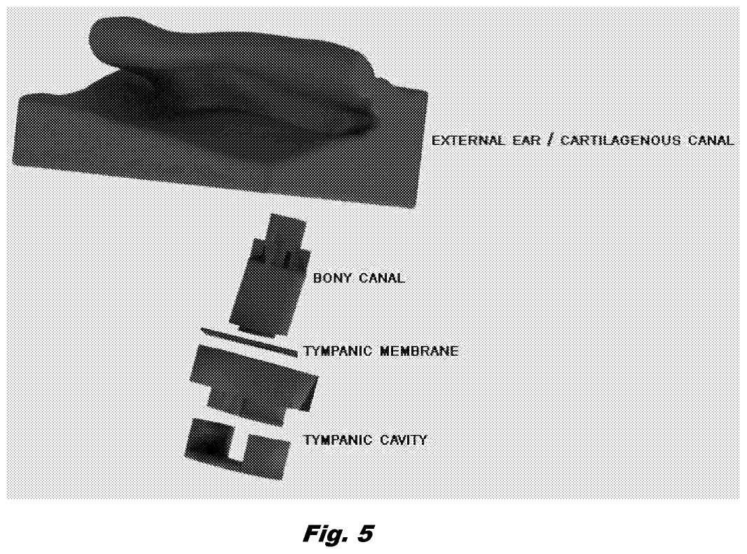

Based on the CT scan measurements, separate models of the medial external canal and middle ear were designed in Fusion 360 (Autodesk Inc, San Rafael, Calif.) (FIG. 5). The medial canal section articulated with the 3D print of the external ear and cartilaginous canal. A separate articulation between the medial canal and tympanic cavity incorporated the reusable tympanic membrane.

The 3D printed modules were created on an Object 260 Connex3 (Stratasys, www.stratasys.com) printer. The auricle, periauricular facial tissue and cartilaginous external auditory canal were printed in Tango+ polymer. The bony external canal and tympanic cavity were printed in Veroclear. Tango+ and Veroclear are proprietary (Stratasys) photopolymers cured with UV light. Tango+ has rubber-like qualities. Veroclear is much harder. The tympanic membrane consisted of a piece of parafilm stretched over the opening of the tympanic cavity module that could be removed and easily replaced after each procedure. Copper tape and conductive paint were used to line the medial external canal and tympanic cavity for the capacitive effectors.

### Sensors, Computer Hardware and Software

MT Sim incorporates capacitive sensors in the medial external auditory canal and walls of the tympanic cavity. The sensor surfaces communicate with a microcontroller that detects instrument contact. The sensor surface can detect any contact with an electrical conductor and is very sensitive to the instruments used during myringotomy and tympanostomy tube insertion.

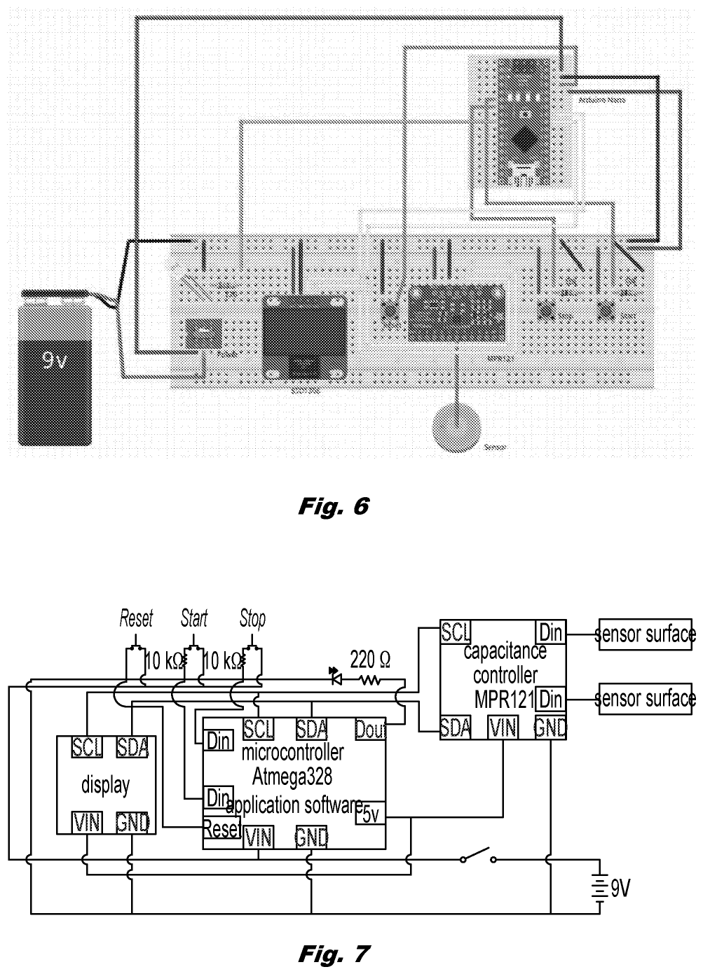

The processing unit can include an ATmega 328 microcontroller and a 12-key Adafruit MPR121 capacitive sensor breakout (sub-circuit). Sensors communicate with the microcontroller via the breakout. Running time and sensor contact time are displayed on a LCD screen. Additional microcontroller connections control procedure start and stop times and reset the system. FIG. 6 and FIG. 7 show the hardware and basic circuit configurations for the sensor, microcontroller and display systems.

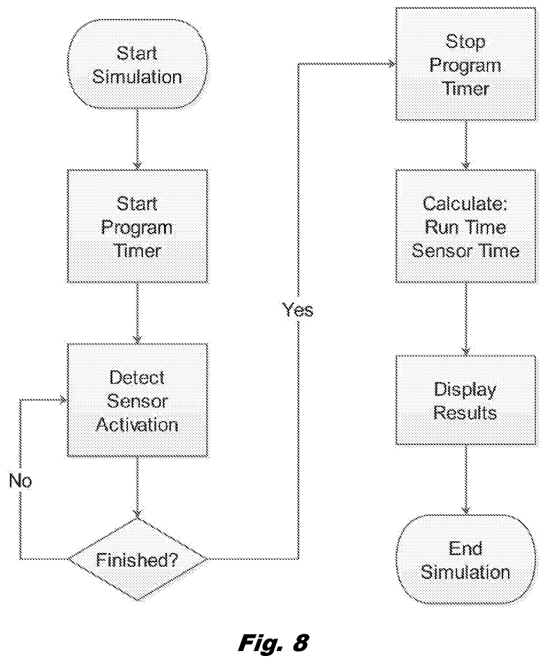

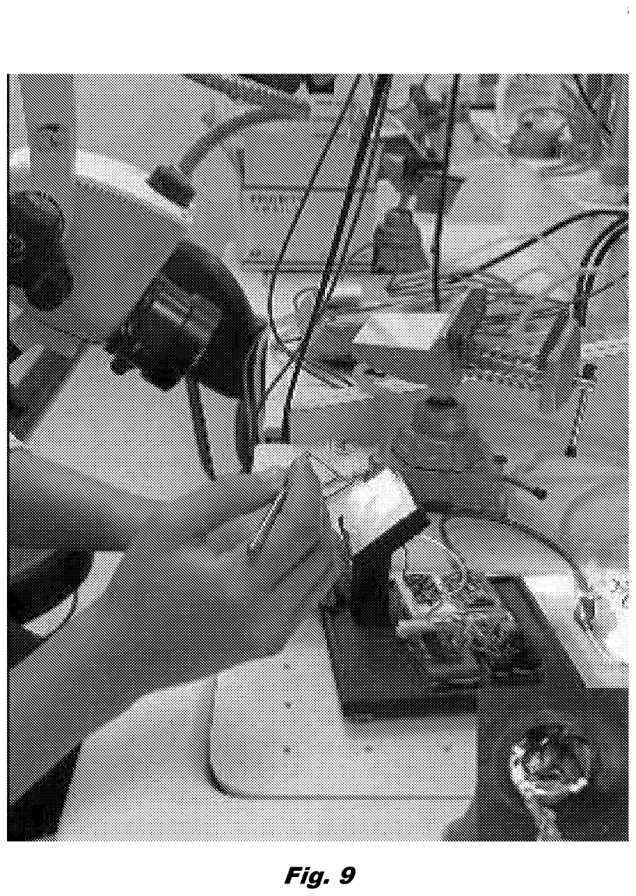

The procedure timing, sensor tracking, and output display are managed by an embedded program written in C++. FIG. 8 shows the program flow. At the end of the procedure, the computer saves the total run time and total sensor contact time.

### Simulator Validation

System validation (construct validation—see discussion section) was performed by evaluating total operating times and sensor contact times. Two groups of users were studied, otolaryngology faculty (n=4) and residents (n=9). Each participant performed 3 simulations for a total of 39 data points (12 faculty and 27 residents). A two tailed t-test (two samples, unequal variances) was used to compare the means for each group. A p-value of 0.05 was considered significant. The run time and sensor time for failed insertion were set at 360 seconds and 60 seconds, respectively.

### RESULTS

### Simulator Function

The sensing system reliably detected standard otologic instrument contact with the medial external canal and tympanic cavity. The microprocessor circuitry and software accurately measured total procedure time and instrument contact time. Initial evaluations by residents and faculty indicate that the system realistically simulates the myringotomy with tympanostomy tube insertion procedure. System usage is shown in FIG. 9.

### Preliminary Simulator Validation

Validation results based on procedure time and sensor contact time are shown in table III. The small sample size affected validation findings. Despite a difference of 10.95 seconds, the mean sensor time contact was not significantly different between the two groups. A significant difference was noted in the operative times.

TABLE I
Simulator validation

| | Faculty | Residents | Difference | p-value |
|---|---|---|---|---|
| Mean Run Time (sec) | 68.56 | 120.77 | 52.22 | 0.04 |
| Mean Sensor Time (sec) | 9.72 | 20.67 | 10.95 | 0.11 |
| Failed Insertions | 0 | 5 | 5 | |

### DISCUSSION

### Surgical Training and Simulation

According to the National Center for Biotechnology Information database, more than 250,000 patients die every year due to medical errors, which makes it the third leading cause of death in the world.¹² Many of these errors lead to preventable surgical complications. Simulation in surgical education can significantly reduce the risks of unsafe care. Allied Market Research valued the global medical simulation market at $986 million in 2016. They expect this to reach $2.5 billion by 2023.¹³

During the early 20th century, William Hallstead defined the traditional apprenticeship model for surgical education.¹⁴ It became the standard for surgical residency training. With advances in digital technology including visual computer systems and 3-D printers, surgical simulation is increasingly used to reduce training times and improve patient outcomes. A broad range of simulated surgical procedures now exist in otolaryngology.¹⁵ In 2017, Javia and Sardesai¹⁶ reviewed the state of surgical simulation in otolaryngology. They classified simulators according to the device construction (physical model versus virtual reality) and the procedure being simulated. Simulators developed for otolaryngology include physical and virtual reality models for otoscopy, myringotomy with tube insertion, stapedectomy, mastoidectomy and endoscopic ear surgery.¹⁷ Some of the models are constructed from readily available components (syringes, surgical glove material). Three-dimensional printed parts are increasingly used for simulator construction.¹⁸

### Mechanical Properties of Human Tissue and Surgical Simulator Design

The physical properties of materials used to create a surgical simulator should match the corresponding tissues as closely as possible. The elastic modulus (Young's modulus), E, is a measure of a material's resistance to displacement or elasticity. It is defined as the ratio of the stress to the strain measured when a known force is used to stretch a sample of the material with known dimensions^{19, 20}. The value varies with the degree of deformation and can be measured for tension or compression. Substances with a high elastic modulus are less elastic. A low value implies greater flexibility. The hardness of materials, including human tissue, is often reported as Shore hardness as measured with a Shore durometer. Shore A is a measure for rubber-like material and Shore D is a scale used for harder materials. Both scales range from 0 (softer) to 100 (harder). The elastic modulus and hardness determine the “feel” of tissue during surgery. Table IV lists the elastic modulus and Shore hardness for several human tissues and the 3D print materials used for simulation in this project.^{21, 22, 23, 24, 25}

TABLE II
Elastic modulus, E, and durometer hardness
of tissues and simulation materials.

| | Eₜᵢₛ | | | Eₛᵢₘ | |
|---|---|---|---|---|---|
| Tissue | (mPa) | Hardness | Simulation | (mPa) | Hardness |
| Auricular | 1 | 20 A | Tango+ | 1.5 | 27 A |
| Cartilage | | | | | |
| Bony Canal/ | 15,000 | 90 D | Veroclear | 2500 | 86 D |
| Tympanic Cavity | | | | | |
| Tympanic | 45 | 10 A | Parafilm | 45 | 15 A |
| Membrane | | | | | |

The simulated tympanic membrane requires replacement after each procedure. The human tympanic membrane is approximately 0.1 mm thick^{28, 27}. Parafilm measures 0.13 mm in thickness and was selected for the tympanic membrane material because of its thickness, physical properties and low cost.

### Sensor Technology

This device incorporates sensing technology that quantitatively tracts the user's ability to accurately place the surgical instruments. Capacitive sensors detect instrument contact with anatomical structures that are potentially injured during live surgery.

Capacitance is a measure of a circuit's ability to store charge on a per volt basis. A capacitive sensor comprises a resistor-capacitor circuit (RC circuit) that detects the change in capacitance in an electric field between two charged plates due to the influence of an external conductor²⁸.

The change in capacitance is proportional to the conductivity and size of the external conductor (a surgical instrument in this case). The plates become polarized as their charges reach equilibrium with the source. Typically, a larger plate is connected to ground and is shielded from external contact. The smaller surface is exposed to external touch. When an external conductor contacts the exposed charged surface, the total capacitance of the circuit increases.

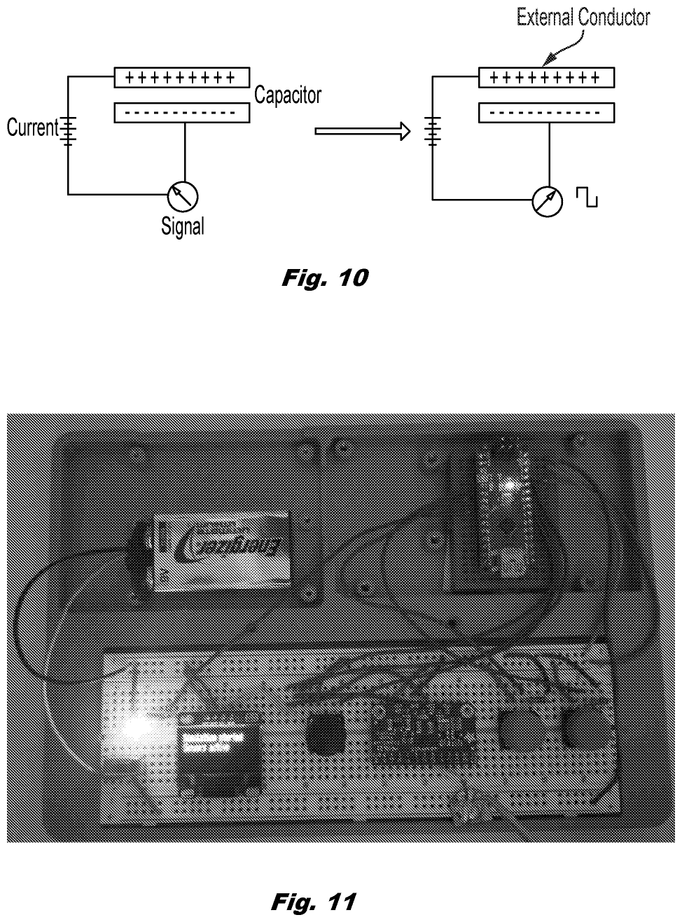

Capacitance based sensing systems are very sensitive and capable of detecting minimal force applied at pin-point areas of the sensing surface. Additional advantages include simple calibration and stability over a wide range of operating temperatures. FIG. 10 shows the conceptual design for a capacitive sensor.

Engineers use capacitive sensing technology in applications to detect proximity, displacement or force. They are commonly used in touchscreens for input in smart phones and tablet computers.²⁹ The external conductive plate of the sensor does not need to be a single planar surface. In our case, the surfaces of complex anatomical structures were coated with conductive material that was wired to the sensor circuit. Additional circuitry and software measure the change in capacitance and report contact with the sensor.

Our system incorporates a self-contained scoring system that tracks operator efficiency as well as the accurate placement of instruments measured by the sensing system. Efficiency is important during tympanostomy tube placement because of the potential consequences of prolonged anesthesia in young children. It is also a commonly performed, high volume procedure that impacts overall operating room efficiency.

### Validation

Central to the deployment of surgical simulation is the concept of the validity framework. Validation is a measure of simulator realism. The theoretical basis for the assessment of surgical skills requires a validation framework that is uninfluenced by observer bias.³⁰ Most studies of surgical simulators correlate models with in-vivo procedures in purely descriptive terms. Validation attempts to classify the model in term of its fidelity to the simulated procedure and effectiveness relative to other means of surgical training.

Well defined criteria for surgical simulator validation exist in the literature³¹ but are not uniformly applied. During the design, simulator engineers look carefully at the response process to ensure that the simulation follows the steps performed during live surgery. Systematic rating scales are available to grade the validation process.³²

Face validity is a subjective assessment of the simulator's realism. It reflects the model's anatomical accuracy and the fidelity of the simulated tissue types.³³ Content validity is also a subjective assessment of the simulated procedure relative to the actual procedure (i.e. are the instruments the same, is the positioning of the patient the same, etc.).³⁴ Construct validity is a quantitative or subjective measure of success when performing the procedure that should improve with increasing operator experience or expertise.^{35, 36} Transfer validity measures the simulator's ability to improve the operator's performance during the live procedure. Published studies either have not mentioned transfer validity or have only discussed it in qualitative terms. Concurrent validity compares the simulator to traditional methods of surgical training (e.g. observation and hands-on performance with attending supervision). Again, existing studies of surgical simulation have only addressed this in qualitative terms.

Validation frameworks are evolving from simple observational studies to statistically verified measures of operator performance. A surgical simulator does not require all forms of validation to be useful. Table V summarizes the types of validation for surgical simulators. Our system incorporates quantitative measures of operator proficiency (run time and instrument accuracy) that will enable comparisons between user groups and between the simulator and the live surgical procedure.

TABLE III
Summary of formal surgical simulator validation
methods described in the literature.

| Validation | Performance Parameter | Measurement |
|---|---|---|
| Face | Anatomical accuracy | Subjective rating scale |
| Content | Fidelity to in vivo procedure | Subjective rating scale |
| Construct | Operator experience/ | Quantitative or subjective |
| | expertise | rating |
| Transfer | In vivo proficiency | Subjective rating scale |
| Concurrent | Performance relative to | Subjective rating scale |
| | traditional training | |

### Additional Embodiments

Without wishing to be bound by theory, a surgical simulator will never completely replicate the experience of working with living human tissue. Embodiments that approach replication can include 3D printed and moldable materials, including silicon, to improve the look and feel of the soft tissues. The surgical feel of the tympanic membrane during myringotomy and component dimensions can be manipulated to achieve greater realism.

Capacitance sensing technology can be used in any simulator where errant instrument placement is important. Without being bound by theory, the sensing technology presently disclosed can be applied in endoscopic sinus, skull base and endoscopic vocal fold surgery.

### CONCLUSIONS

Surgical simulation is evolving along two pathways—physical modeling and virtual reality. MTSim is the first physical based model to incorporate capacitance sensing technology to measure instrument accuracy and software to objectively assess operator proficiency. Subjective user assessment and initial system validation indicate that the simulator can improve trainee performance for myringotomy and tympanostomy tube insertion.

### REFERENCES CITED IN THIS EXAMPLE

17. Rosenfeld R M, Schwartz S R, Pynnonen M A, et al. Clinical practice guideline: tympanostomy tubes in children. Otolaryngology—Head and Neck Surgery. 149(1suppl), S1-S35.
18. McLelland C A. Incidence of complications from use of tympanostomy tubes. Arch Otolaryngol. 1980; 106(2): 97-99.
19. Montague M L, Lee M S W, and Hussain S S M. Human error identification: an analysis of myringotomy and ventilation tube insertion. Arch Otolaryngol Head Neck Surg. 2004; 130(10): 1153-1157.
20. Sutherland L M, Middleton P F, Anthony A, et al. Surgical simulation: a systematic review. Ann Surg. 2006; 243(3): 291-300.
21. Schout B M, Hendrikx A J, Scheele F, et al. Validation and implementation of surgical simulators: a critical review of present, past, and future. Surg Endosc. 2010; 24: 536.
6. Moglia A, Vincenzo F, Morelli L, Ferrari M, et al. A systematic review of virtual reality simulators for robot-assisted surgery. European Urology. 2016; 69(6): 1065-1080.
7. McCloy R and Stone R. Science, medicine, and the future. Virtual reality in surgery. BMJ. 2001; 323(7318): 912-915.
8. Barsom E Z, Graafland M and Schijven M P. Systematic review on the effectiveness of augmented reality applications in medical training. Surg Endosc. 2016; 30: 4174.
9. Brewin J, Nedas T, Challacombe B, et al. Face, content and construct validation of the first virtual reality laparoscopic nephrectomy simulator. BJU International. 2010; 106: 850-854.
10. Huang C., Agrawal S K. and Ladak H M. Virtual reality simulator for training in myringotomy with tube placement. J. Med. Biol. Eng. 2016; 36: 214.
11. Malik H H, Darwood R J, Shaunak S, et al. Three-dimensional printing in surgery: a review of current surgical applications. Journal of Surgical Research. 2015; 199(2): 512-522.
12. Anderson J G and Abrahamson K. Your Health Care May Kill You: Medical Errors. Stud Health Technol Inform. 2017; 234: 13-17.
13. Medical Simulation Market. Allied Market Research.
14. Kerr B and O'leary J P. The training of the surgeon: Dr. Halsted's greatest legacy. The American Surgeon. 1999: 65(11): 1101-2.
15. Musbahi O, Aydin A, Al Omran Y, et al. Current status of simulation in otolaryngology: a systematic review. Journal of Surgical Education. 2017; 74(2): 203-215.
16. Javia L and Sardesai M D. Physical models and virtual reality simulators in otolaryngology. Otolaryngol Clin N Am. 2017; 50: 875-891.
17. Wiet G J, Sorensen M S and Andersen S A. Otologic skills training. Otolaryngol Clin N Am. 2017; 50: 933-945.
18. VanKoevering K K and Malloy K M. Emerging role of three-dimensional printing in simulation in otolaryngology. Otolaryngol Clin N Am. 2017; 50: 947-958.

### 19. Enderle J, Bronzino J and Blanchard S. Introduction to Biomedical Engineering: Edition 2. Elsevier, 2005.

20. Roy R, Kohles S S, Zaporojan V, et al. Analysis of bending behavior of native and engineered, auricular and costal cartilage. Proceedings of the IEEE 27th Annual Northeast Bioengineering Conference (Eds. J D Enderle, L L Macfarlane). 2001: 31-32.
21. Kuru I, Maier H, Muller M, et al. A 3D-printed functioning anatomical human middle ear model. Hearing Research. 2016; 340: 204-213.
22. Luo H, Dai C, Gan R Z and Lu H. Measurement of Young's modulus of human tympanic membrane at high strain rates. J Biomech Eng. 2009; 131(6): 064501. doi: 10.1115/1.3118770.
23. Tango+ datasheet. Stratasys Corporation. https://www.stratasys.com/-/media/files/material-spec-sheets/mss_pj_tango_0318a.pdf. Accessed Feb. 8, 2019.
24. Parafilm M datasheet. Sigma Aldrich Corp.
25. Veroclear datasheet. Stratasys Corporation.
26. Van der Jeught S, Dirckx J J J, Aerts J R M, et al. Full-field thickness distribution of human tympanic membrane obtained with optical coherence tomography. JARO. 2013; 14: 483.
27. Berdich K N, Faur N, Gentil F, et al. Biomechanical study of myringotomy through simple incision and drainage tube insertion. 2013 E-Health and Bioengineering Conference (EHB). 2013: 1-4.
28. Osoinach B. Proximity capacitive sensor technology for touch sensing applications.
29. Hu X and Yang W. Planar capacitive sensors—designs and applications. Sensor Review. 2010; 30(1): 24-39.
30. Borgersen N J, Naur T M, Sorensen S M, et al. Gathering validity evidence for surgical simulation: a systematic review. Annals of Surgery. 2018; 267(6): 1063-1068.
31. Van Nortwick S S, Lendvay T S, Jensen A R, et al. Methodologies for establishing validity in surgical simulation studies. Surgery. 2010; 147(5): 622-630.
32. Ghaderi I, Manji F, Park Y S, et al. Technical skills assessment toolbox: a review using the unitary framework of validity. Annals of Surgery. 2015; 261(2): 251-262.
33. Sowerby L J, Rehal G, Husein M, et al. Development and face validity testing of a three-dimensional myringotomy simulator with haptic feedback. Journal of Otolaryngology—Head & Neck Surgery. 2010; 39(2): 122-129.
34. Huang C, Cheng H, Bureau Y, et al. Face and content validity of a virtual-reality simulator for myringotomy with tube placement. Journal of Otolaryngology—Head & Neck Surgery. 2015; 44: 40.
35. Hong P, Webb A N, Corsten G, et al. An anatomically sound surgical simulation model for myringotomy and tympanostomy tube insertion. International Journal of Pediatric Otorhinolaryngology. 2014; 78(3): 522-529.
36. Volsky P G, Hughley B B, Peirce S M and Kesser B W. Construct validity of a simulator for myringotomy with ventilation tube insertion. Otolaryngology—Head and Neck Surgery. 2009;141(5):603-608.

### Example 2

### Description of the Exemplary Technology

In an embodiment, the system is a design for tracking instrument placement accuracy during procedures performed on a bench model surgical simulator. It can incorporate a capacitance sensor that can detect contact with a surgical instrument and can be adapted to any surface in a simulated surgical field. Multiple sensor surfaces on a single simulator can be monitored.

### Hardware

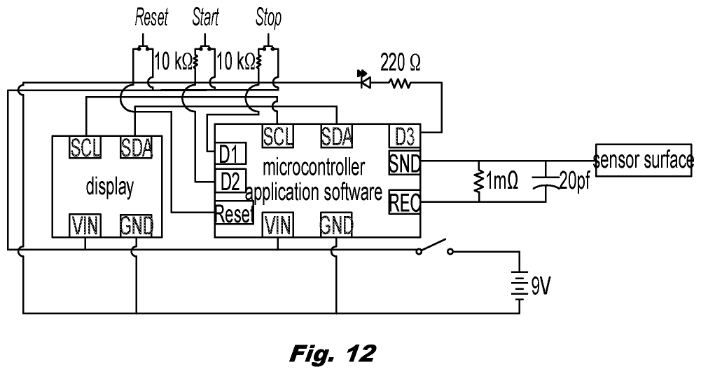

The processing unit comprises a microcontroller and a capacitive sensor subcircuit. The sensors are any connected conductive surfaces on the simulator and communicate with the microcontroller via the subcircuit. Running time, sensor contact time and a final score are displayed on an LCD display. Additional microcontroller connections control procedure start/stop times and reset the system. FIGS. 6, 11, and 12 show an exemplary prototype and circuit schematic for the sensor and scoring system.

### Software

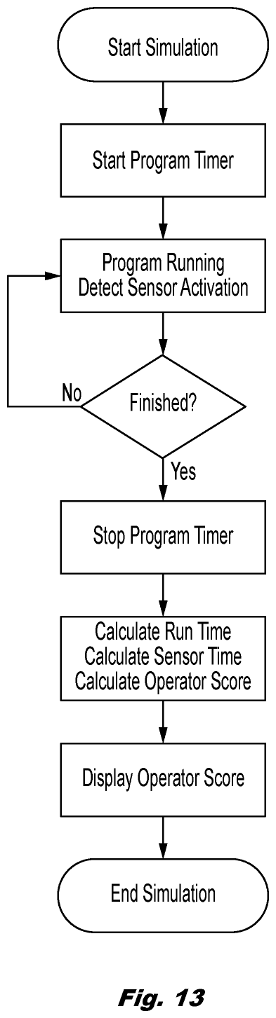

The procedure timing, sensor tracking, score calculation and display can be managed by an embedded program written in C++. FIG. 13 shows the program flow. At the end of the procedure, the computer saves the total run time and total sensor contact time and calculates the user's score. The microcontroller is programmable. The software and scoring algorithm can be updated via a USB connection or other suitable means known in the art.

### Scoring

The scoring algorithm is closely linked to the sensing system. It assesses operator efficiency and accuracy. The system scores these operator attributes with the procedure run time and sensor contact time, respectively.

One exemplary embodiment employs the following scoring system:

$$run\_score = ( allocated\_time - run\_time ) \times run\_scale$$

$$sensor\_penalty = sensor\_time \times sensor\_scale$$

$$final\_score = run\_score - sensor\_penalty$$

The allocated time will vary depending on the simulated procedure and can be determined by surgical experts who perform the procedure in vivo. The scale factors are determined empirically. The final score is truncated to the range 0-100 where higher scores indicate greater proficiency.

### Commercial Applications of the Technology

The technology can be employed in any bench model surgical simulator to detect instrument contact with surfaces in the surgical field. This includes applications in general surgery, otolaryngology, neurosurgery, gastroenterology, urology and cardiovascular surgery.

### Non-limiting Advantages of the Currently Disclosed Systems and Methods

The system innovates the use of surgical instrument tracking to objectively and quantitively measure surgeon proficiency. It will reduce surgical error, decrease operating times and improve surgical outcomes. Capacitance based sensing systems are very sensitive and capable of detecting minimal force applied at pin-point areas of the sensing surface. A wide range of conductive materials can be used in the simulated surgical field to detect instrument contact. The system has been tested with copper foil, conductive cloth and conductive paint. Any monitored surface is easily connected to the controller by a single wire. Additional advantages of the capacitance-based system include simple calibration and stability over a wide range of operating temperatures. The integrated scoring system provides immediate feedback to the operator who is often a resident surgeon or medical student. The quantitative measure of operator proficiency also facilitates simulator validation.

### Example 3

### Introduction/Background

Beyond basic soft tissue handling and suturing, tympanostomy tube insertion is one of the first surgical skills acquired by otolaryngology residents. Otologic surgery is technically challenging and trainees often struggle to gain proficiency working in the ear canal through a microscope. Inexperienced surgeons are more likely to cause inaccurate tube placement, canal injury, troublesome bleeding and prolonged anesthesia. Efficient surgical tympanostomy tube placement requires practice.

In this disclosure, we describe a bench model device engineered for the realistic simulation of myringotomy with tympanostomy tube insertion performed using a standard operating microscope. A particular innovative feature is the integration of a sensing system to detect instrument placement accuracy. We incorporate scoring software to grade the user and validate the system.

In an embodiment, the system can include the following components:

22. 3D printed auricle, external auditory canal, tympanic cavity, and peri-auricular soft tissue.
23. A capacitive sensor system integrated with the bony external canal and tympanic cavity that detects instrument contact.
24. A programmable microcontroller and supportive electronics with a timer and sensor interface.
25. Housing for the system hardware.
26. Software to monitor the operating time, detect sensor interactions and compile the user's score.

### Study Population

Model validation scores will be calculated from quantitative measures of operator efficiency and accuracy. The operator cohort includes students, otolaryngology residents and otolaryngology faculty.

### Objective Outcomes

1. Develop a model for practicing myringotomy with tympanostomy tube insertion.
2. Construct a surgical simulator with instrument sensing technology.
3. Analyze improvement in surgical skill and technique among trainees after practice with the simulator.

We claim:

1. A surgical simulator comprising:
a surgical replica configured to approximate a surgical tissue or a surgical field;
a capacitance sensor with at least one sensor surface;
a processing system communicatively linked to the capacitance sensor and configured to provide an operator proficiency score; wherein
the surgical simulator is configured to simulate a surgical procedure and assess an operator's proficiency with the surgical procedure.

2. The surgical simulator of claim 1, wherein the sensor surface is configured to detect contact with an electrical conductor.

3. The surgical simulator of claim 2, wherein the electrical conductor comprises a surgical instrument.

4. The surgical simulator of claim 1, wherein the surgical simulator is configured to simulate surgeries in the field of general surgery, otolaryngology, neurosurgery, gastroenterology, urology, cardiovascular surgery, oral surgery, pediatric surgery, plastic surgery, orthopaedic surgery, or cardiothoracic surgery, dentistry, podiatry, or any a combination thereof.

5. The surgical simulator of claim 1, wherein the surgical procedure comprises myringotomy, tympanostomy tube insertion, endoscopic sinus surgery, skull base surgery, laryngeal surgery other types of ear surgery or a combination thereof.

6. The surgical simulator of claim 1, wherein the at least one sensor surface comprises a copper foil, a conductive cloth, conductive paint, or a combination thereof.

7. The surgical simulator of claim 1, wherein the at least one sensor surface is at least partially integrated within or coated upon the surgical replica.

8. The surgical simulator of claim 1, further comprising a display screen, wherein the display screen is configured to display the operator proficiency score.

9. The surgical simulator of claim 1, wherein the surgical replica comprises an artificial human ear.

10. The surgical simulator of claim 9, wherein the artificial human ear comprises a middle ear section and an outer ear section.

11. The surgical simulator of claim 10, further comprising an auricle, an external auditory canal, a tympanic cavity, a tympanic membrane, or a combination thereof.

12. The surgical simulator of claim 11, wherein the external auditory canal further comprises a cartilaginous-type canal, a bony-type canal, or a combination thereof.

13. The surgical simulator of claim 11, wherein the at least one sensor surface is integrated within the auricle, the external auditory canal, the tympanic cavity, the tympanic membrane, or a combination thereof.

14. The surgical simulator of claim 11, wherein the tympanic membrane is configured to be replaced between simulations.

15. The surgical simulator of claim 11, wherein the tympanic membrane comprises a thickness of about 0.5 mm to about 1.5 mm.

16. The surgical simulator of claim 15, wherein the tympanic membrane comprises a flexible film that further comprises wax and polyolefins.

17. A method of simulating a surgical procedure, the method comprising:
simulating a surgical procedure using the surgical simulator of any one of claims 1-16;
determining the total amount of time required to complete the surgical procedure;
determining the total amount of sensor contact time, wherein sensor contact time comprises the amount of time that an electrical conductor was in contact with the sensor surface during the surgical procedure; and
providing an operator proficiency score.

18. The method of claim 17, wherein the proficiency score is inversely proportional to the total amount of time required to complete the surgical procedure, the total amount of sensor contact time, or a combination thereof.

19. The method of claim 17, further comprising displaying the operator proficiency score on a display screen.

20. A system for simulating a surgical procedure, the system comprising:
the surgical simulator of any one of claims 1-16;
software configured to calculate the operator proficiency score; and
a display screen configured to display an operator proficiency score.

21. The system of claim 20, wherein the system is configured to determine running time and sensor contact time.

22. The system of claim 20, further comprising a microcontroller, wherein the microcontroller comprises a timer, an interface to the sensors, an output to the display, or a combination thereof.

23. The system of claim 21, wherein the microcontroller is configured to detect the total time required to complete the surgical procedure, the number of contacts between an electrical conductor and the sensor surface, the total amount of time that the electrical conductor contacts the sensor surface, or a combination thereof.

24. The system of claim 21, wherein the microcontroller is configured to control a procedure start time, to control a procedure stop time, reset the system, or a combination thereof.

25. The system of claim 20, wherein the system is configured to track instrument placement accuracy.
